(19) United States
(12) Patent Application Publication
(10) Pub. No.: US 2024/0060586 A1
(43) Pub. Date: Feb. 22, 2024

(54) SIZE-SPECIFIC TUBING CONNECTOR ASSEMBLY

(71) Applicants: ASIF K. MUSTAFA, WILLOWNROOK, IL (US); DEVANG J. JOSHI, CAROL STREAM, IL (US)

(72) Inventors: ASIF K. MUSTAFA, WILLOWNROOK, IL (US); DEVANG J. JOSHI, CAROL STREAM, IL (US)

(21) Appl. No.: 17/891,499

(22) Filed: Aug. 19, 2022

(51) Int. Cl. F16L 33/30

(52) U.S. Cl. CPC F16L 33/30

(57) ABSTRACT

This invention relates to a size-specific tubing connector assembly used for selectively connecting tubing segments of various inner diameters. A central housing defines at least one through bore between at least first and second ends configured for axial removable engagement with at least a first pair of a plurality of tubular fitments. Each fitment of the at least first pair defines a common inner and outer diameter and is configured for axial alignment in opposition with one another about the housing in series relation to define fitment series “A” and “B” at respective ends of the housing, with the fitments of each series “A” and “B” arranged preferably in a descending order of the respective fitment's outer diameter.

### CROSS REFERENCE TO RELATED APPLICATIONS

None

### TECHNICAL FIELD OF THE INVENTION

This invention relates to conical tubing connectors. More specifically, this invention relates to a size-specific tubing connector assembly used for selectively connecting tubing segments of various inner diameters.

### BACKGROUND OF THE INVENTION

Plastic tubing is often used in medical and surgical settings to perform various fluid-flow functions and connect various components in fluid-flow communication with one another. For example, such tubing can carry vital fluids into or out of a patient, such as oxygen, blood, and/or saline or various medicinal fluids. The tubing may also perform various other fluid-flow functions, to include feeding and/or drainage functions.

As such, a wide variety of tubing sizes is utilized in these settings, with such tubing often varying in both length and inner diameter. However, due to the variety of tubing sizes utilized in these settings, complications may arise in connecting the tubing to the various components and/or other tubing utilized in a medical or surgical setting. To facilitate such connection, various connectors are present in the prior art that are conical in structure and staged in varying diameter along their length. FIG. 1 illustrates one such embodiment of a staged, conical connector.

As illustrated in FIG. 1, the prior art connector comprises a unitary, ferrule structure defining opposing conical portions of varying outer diameter. The varying outer diameters of the opposing conical portions are staged to increase along the length of the ferrule such that the smallest outer diameters are defined at the ferrule's outer ends and the larger outer diameters are defined inwardly of one another, culminating in a single outer diameter at a midpoint of the ferrule. Thus, when connecting segments of tubing with the connector, the connector is inserted into an end of the tubing and forced inwardly such that the tubing's inner circumferal surface makes frictional contact with the outer circumferal surface of a stage of the connector having an outer diameter of approximately the same size as that of the tubing inner circumferal surface. Depending on the size of the tubing's inner diameter, the frictional contact may occur at a stage of the connector located at the connector's outer end, or at one of the inwardly located stages of larger outer diameter defined towards the connector's midpoint.

Although such prior art connectors may indeed facilitate the connection of one or more tubing segments or other components in fluid communication with one another, they nonetheless present numerous disadvantages within the prior art. These disadvantages all originate from the fact that a single connector is often utilized to connect a wide variety of tubing segments to one another, regardless of the inner diameters defined by the tubing. Because of the wide variety of tubing inner diameters present in a medical or surgical setting, the “one-size-fits-all” approach of the single connector often proves inadequate in achieving a correct and fluid-tight connection between the tubing segments. An inadequate fit of the components with one another can result in them coming apart; thus, dangerously interrupting the fluid-flow there-through. This inadequacy is often exacerbated in a medical or surgical setting where time is of the essence in performing a given procedure, and the connector itself or the tubing must be replaced with other sized components until a fluid-tight connection is achieved.

Yet other disadvantages present themselves where medical practitioners attempt to connect the tubing segments and a prior-art connector, via the force-fit of the components with one another, in the presence of blood or other bodily fluids often present in a medical or surgical setting. The presence of these slippery fluids on the tubing and/or connector creates problems with gripping the components adequately enough to force-fit the components together into a fluid-tight configuration. An inadequate force-fit of the components with one another can again result in them coming apart; thus, again dangerously interrupting the fluid-flow there-through.

Furthermore, even if a fluid-tight connection is achieved, further disadvantages present themselves in terms of achieving efficient fluid-flow through the connected tubing. More specifically, if the inner circumferal surface of the tubing makes a friction fit with the inwardly located stages of the connector such that those stages located at the connector's outer ends extend needlessly into the interior of the tubing, flow voids are created between the inner circumferal surface of the tubing and outer circumferal surface of those stages of the connector not in fitment therewith. These voids may result in a disadvantageous clogging of the flow within the tubing when the fluid flowing there-through comprises blood (i.e., during ECMO configurations) or bodily waste products (i.e., during wound or digestive tract draining configurations).

Also, because of the unitary structure of prior art connectors, generally comprised of materials more rigid than the tubing segments they connect, a dangerous kinking of the tubing is prone to occur if the tubing is bent at a location proximal to the outer ends of the connector itself. This is especially true where the tubing segments are of extended length and the weight of the tubing acts against the connector, thus kinking the tubing at an end of the connector and interrupting the fluid-flow there-through. The rigidity of the connector assembly also precludes it from axially extending or contracting to accommodate its connection with tubing segments of extended or insufficient length. More specifically, if one or more of the tubing segments are not long enough to connect to a prior-art connector, the rigid body of such a connector cannot extend in length to accommodate for the insufficient length of the tubing segment(s). Similarly, if one or more of the tubing segments are too long when connecting to the prior-art connector, the rigid body of such a connector cannot contract in length to accommodate for the extraneous length of the tubing segment(s). Even where the aforementioned kinking does not occur, extended lengths of tubing connected by a connector are often not properly supported, thus resulting in the tubing undesirably becoming located on the floor or other areas prone to dirt or germs.

Thus, what is needed is a tubing connector sized for appropriate connection with tubing segments defining a given inside diameter while avoiding a unitary, “one-size-fits-all” structure prone to an inadequate fluid-tight connection with tubing of various inside diameters. The connector should facilitate a connection between tubing segments without having outer ends needlessly protruding into the tubing's interior. The connector should facilitate the connection of tubing segments while preventing an occurrence of kinking within the tubing itself, as well as accommodating its connection with tubing segments of extended or insufficient length. The connector should also facilitate a support of the tubing to prevent it from becoming located in areas prone to dirt and germs. The connector should further facilitate the infusion of liquids into, or the extraction of fluids from, the tubing. The present invention satisfies the foregoing needs while presenting other advantages over the prior art as well.

Although the foregoing disadvantages are described in relation to medical or surgical applications, it is understood that the advantages provided by the present invention are equally applicable to non-medical and/or non-surgical applications as well. For example, the present invention may be configured for use with common household garden hoses and/or plumbing applications.

### SUMMARY OF THE INVENTION

This invention relates to conical tubing connectors. More specifically, this invention relates to a size-specific tubing connector assembly used for selectively connecting tubing segments of various inner diameters. In a first embodiment of the tubing connector assembly, a central housing defines at least one through bore between at least first and second ends configured for axial removable engagement with at least a first pair of a plurality of tubular fitments. Each fitment of the at least first pair defines a common inner and outer diameter and is configured for axial alignment in opposition with one another about the housing in series relation to define at least a pair of fitment series, namely, series “A” and “B” located at respective opposing ends of the housing, with the fitments of each series “A” and “B” arranged preferably in a descending order of the respective fitment's outer diameter.

Each fitment of each series “A” and “B” is removably engageable with an adjacent fitment and configured for fluid communication therewith, with the first pair also configured for fluid communication with the housing. A second pair, third pair, fourth pair, fifth pair, sixth pair, seventh pair or any quantity of fitment pairs may be utilized with the housing. It is further understood that any combination of fitments may be utilized with the housing as well, to include non-paired fitments. Yet alternatively, one or more pairs and/or one or more single fitments may be engaged in series with one or more fitments of the first fitment pair. This variety of engagements and combinations facilitates the connection of a variety of tubing segments with one another, regardless of whether or not the tubing segments have common or differing inner diameters. In a preferred embodiment, the central housing and fitments each comprise medical grade plastic preferably manufactured via an injection molding process. However, it is understood that one or more of these components may comprise stainless steel, brass, or various thermo-plasts as well utilizing other manufacturing processes such as machining, extruding, 3-D printing, etc., with these latter compositions more suitable for common household (i.e., connecting garden hoses) or plumbing applications.

The at least one bore of the central housing preferably defines internal threads on an inner circumferential surface of the bore, respectively proximal to the housing's first and second ends. The first pair of the plurality of paired tubular fitments each defines external threads on the fitment's outer circumferential surface at a first end and internal threads on the fitment's inner circumferential surface at a second end. The external threads of each fitment and of the at least first pair are configured for threaded engagement with the internal threads of the housing. Each tubular fitment of the remaining fitments of the series “A” and “B” define respective external threads on each fitment's outer circumferential surface at a first end and internal threads on the fitment's inner circumferential surface at a second end, with the external threads of a given fitment configured for threaded engagement with the internal threads of an adjacent fitment within the series. It is nonetheless understood, however, that the overall reverse common thread arrangement may be utilized by a connector defining external threads at each end of the housing, and each fitment defining internal threads at its first end and external threads at its second end. Each fitment may harbor a gasket/washer that comprises of various materials such as rubber, silicone, plastic etc. allowing for greater fluid-tight seal between engaged fitments.

The first and second outer ends of the central housing preferably define at least one peripheral flange about the housing's outer circumferential surface. Similarly, each tubular fitment preferably defines at least one peripheral flange about the fitment's outer circumferential surface proximal to the end of the fitment defining the internal threads. The at least one peripheral flange of both the housing and fitments is configured for a fluid-tight frictional engagement with the inner circumferential surface of the at least one segment of tubing.

In an alternative embodiment of the fitments of the connector assembly, the outer circumferential surface the housing and/or one or more fitments define a grip between the fitment's respective first and second ends and/or the housing's outer ends. The grip aids in a grasping and rotating of the fitment and/or housing by a users' hand or tool in relation to one another or the at least one tubing segment. A further embodiment of the central housing of the connector defines an axially flexible portion between the housing's first and second ends. This flexible portion allows the central housing to axially bend to alleviate any kinking that may occur within the at least one tubing segment connected to the connector's fitment(s). It also allows the housing to axially extend and/or contract between its outer ends to accommodate a connection of the connector assembly with tubing segments of extended or insufficient length. Other embodiments of the central housing define at least one support on its outer surface. The support facilitates a connection of the connector and attached at least one tubing segment to a hospital bed, IV stand or other component to prevent the connector and tubing segment from becoming contaminated with the floor or some other undesirable surface.

In yet another embodiment, the central housing defines a fluid-tight joint between its outer ends that allows for the housing to be disassembled such that the series of one or more fitments and/or tubular segments may be removably engaged to one or more of the outer ends of the connector during a medical or other application, and thereafter reassembled when the desired configuration of fitment(s)/tubular segment(s) is engaged therewith. A further embodiment of the central housing defines at least one access port through the housing's outer surface in fluid communication with the at least one bore. The at least one access port allows for the infusion of fluids or medications into the connector and at least one tubing segment, or the extraction of blood or other fluids there-from for subsequent testing or laboratory analysis. In yet a further embodiment, the central housing defines a valve to occlude or regulate the axial flow of fluid there-through. The housing may the foregoing components alone or in combination with one or more other components.

Yet a further embodiment of the connector utilizes a central housing defining a “Y” configuration to define first, second and third outer ends of the at least first and second ends. These first, second and third ends of the housing are configured for axial removable engagement with respective first, second and third fitments of the plurality of tubular fitments, with each fitment preferably defining a common inner and outer diameter and again axially aligned with the remaining fitments of the plurality about the housing in the descending order of the fitment's outer diameter. Again, each fitment of the series is removably engageable with an adjacent fitment and configured for fluid communication therewith.

### DESCRIPTION OF THE DRAWINGS

FIG. 1 illustrates a prior art unitary tubing connector;

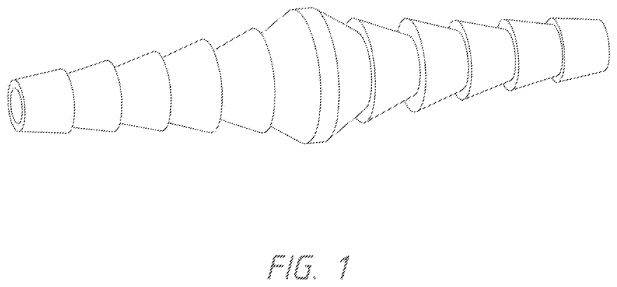

FIG. 2 illustrates a perspective view of one embodiment of the connector;

FIG. 3 illustrates an exploded view of the connector embodiment of FIG. 2;

FIG. 4 illustrates one embodiment of the grips of the connector's fitments;

FIG. 5 illustrates another embodiment of the grips of the connector's fitments;

FIG. 6 illustrates one embodiment of the flexible portion of the housing;

FIG. 7 illustrates another embodiment of the flexible portion of the housing;

FIG. 8 illustrates a support of the housing;

FIG. 9 illustrates a fluid-tight joint of the housing;

FIG. 10 illustrates at least one access port of the housing;

FIG. 11 illustrates the housing defining multiple components of the foregoing embodiments; and

FIG. 12 illustrates the at least one bore of the housing defining a “Y” configuration.

### DESCRIPTION OF THE EMBODIMENTS

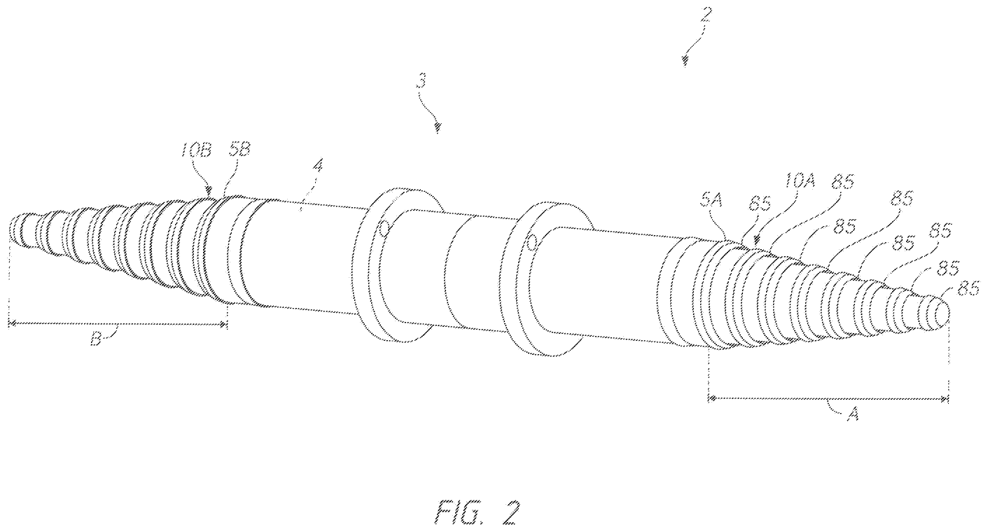

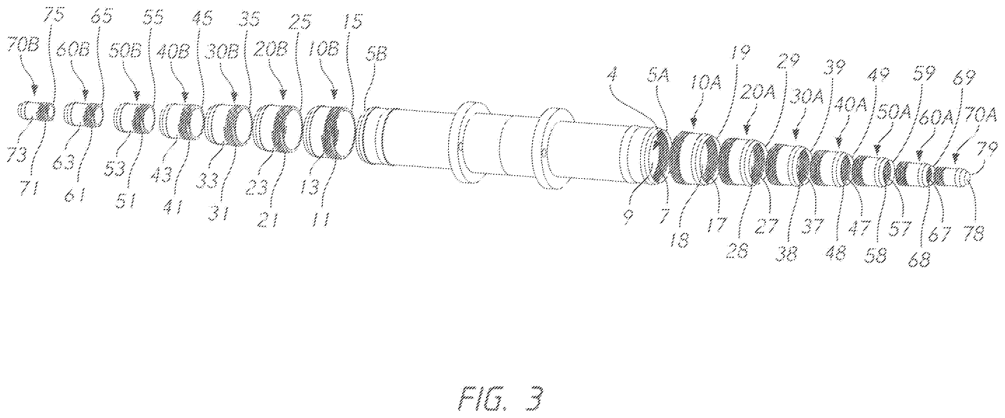

This invention relates to conical tubing connectors. More specifically, this invention relates to a size-specific tubing connector assembly used for selectively connecting tubing segments of various inner diameters. In a first embodiment of the tubing connector assembly 2 illustrated in FIGS. 2 and 3, a central housing 3 defines at least one through bore 4 between at least first and second ends 5A and 5B and configured for axial removable engagement with at least a first pair 10A and 10B of a plurality of tubular fitments. Each fitment 10A and 10B of the at least first pair define inner and outer surfaces 15 and 17 having respective common inner and outer diameters (FIG. 3) and is configured for axial alignment in opposition with one another about the housing 3, in series relation, to define fitment series “A” and “B” at respective ends 5A and 5B of the housing, with the fitments of each series “A” and “B” arranged preferably in a descending order of the respective fitment's outer diameter. Although a descending order of the fitments' outer diameters is preferred to selectively correspond with the given inner diameter of a tubing segment, it is nonetheless understood that the descending order of the fitments may be based upon the fitments' respective outer diameters as well. Each fitment of each series “A” and “B” is removably engageable with an adjacent fitment and configured for fluid communication therewith, with the first pair 10A and 10B also configured for fluid communication with the housing 3.

As more clearly illustrated in FIG. 3, a second pair 20A and 20B, third pair 30A and 30B, fourth pair 40A and 40B, fifth pair 50A and 50B, sixth pair 60A and 60B, and seventh pair 70A and 70B are shown. However, it is understood that any quantity of fitment pairs may be utilized with the housing 3. It is further understood that any combination of fitments may be utilized with the housing 3 as well, to include both paired and/or non-paired fitments. For example, the first fitment pair 10A and 10B may removably engaged with the housing 3, while not having any further fitment pairs engaged therewith. Alternatively, only a single fitment (i.e., 10A or 10B) may be engaged with the housing 3, with or without one or more fitments engaged in series therewith. Yet alternatively, one or more pairs and/or one or more single fitments may be engaged in series with one or more fitments of the first fitment pair 10A and 10B. This variety of engagements and combinations facilitates the connection of a variety of tubing segments with one another, regardless of whether or not the tubing segments have common or differing inner diameters.

In a preferred embodiment, the central housing 3 and fitments each comprise medical grade plastic preferably manufactured via an injection molding process. However, it is understood that one or more of these components may comprise stainless steel, brass, or various thermo-plasts (i.e., polyvinyl chloride; PVC) as well, with these latter compositions more suitable for common household (i.e., connecting garden hoses) or plumbing applications.

Referring again to FIGS. 2 and 3, the at least one bore 4 of the central housing 3 preferably defines internal threads 7 on an inner circumferential surface 9 of the bore, respectively proximal to the housing's first and second ends 5A and 5B. The first pair 10A and 10B of the plurality of paired tubular fitments each define external threads 11 on the fitment's outer circumferential surface 13 at a first end 15 and internal threads 17 on the fitment's inner circumferential surface 18 at a second end 19. The external threads 11 of each fitment 10A and 10B of the at least first pair are configured for threaded engagement with the internal threads 7 of the housing 3. Each tubular fitment of the remaining fitments of the series “A” and “B” (i.e., 20A and 20B; 30A and 30B; 40A and 40B; 50A and 50B; 60A and 60B; and 70A and 70B) define respective external threads (21; 31; 41; 51; 61 and 71) on each fitment's outer circumferential surface (23; 33; 43; 53; 63; and 73) at a first end (25; 35; 45; 55; 65; and 75) and internal threads (27; 37; 47; 57; and 67) on the fitment's inner circumferential surface (28; 38; 48; 58; and 68) at a second end (29; 39; 49; 59; and 69), with the external threads of a given fitment configured for threaded engagement with the internal threads of an adjacent fitment within the series. Each fitment may harbor a gasket/washer abutting a circumferal surface (not shown) defined at one or both of its ends, that comprises of various materials such as rubber, silicone, plastic etc.; thus allowing for a greater fluid-tight seal between engaged fitments.

As illustrated in FIG. 3, fitments 70A and 70B each do not define internal threads on inner circumferential surface 78 at second ends 79. This is because each fitment of this pair preferably comprises a “terminal” fitment configured primarily for engagement with a tubing segment and not with a further fitment of the series. However, it is understood that internal threads (i.e., internal threads 77) may nonetheless be defined therein.

The commonality of the internal/external thread arrangement for the fitment pairs, namely, each fitment defining internal threads at a first end and external threads at a second end is critical for defining a simplified operability of the connector. In a medical or surgical setting, time is of the essence and lives may be at stake when having to select and engage fitments of a connector to accommodate the inner diameter of a given tubing segment. The commonality of the thread arrangement thus serves as a time saving measure in avoiding fumbling with fitments of differing thread arrangement. This commonality is also beneficial in a non-medical or surgical setting, where common homeowners (when connecting garden hose segment) and plumbers (when connecting piping segment) are spared the frustration of “puzzling-together” a connector with fitments having a non-common thread arrangement. It is nonetheless understood, however, that the overall reverse common thread arrangement may be utilized by a connector defining external threads at each end 5A and 5B of the housing 3, and each fitment defining internal threads at its first end and external threads at its second end.

Referring again to FIG. 3, the outer diameter of each fitment within the descending order of the series preferably differs in size from that of an adjacent fitment by an incremental value. Such an incremental value serves to accommodate a connection of the connector's fitment with a wide variety of inner diameters of the at least one tubing segment. Thus, when such fitments are used within a surgical or medical setting, the incremental value is at least one French (Fr.). In other applications of the fitment, to include household or commercial plumbing applications, the incremental value is at least one mm or at least 1/16 of an inch. Moreover, to accommodate the incremental difference between fitments within a series, the outer diameters of the fitments preferably fall within a desired range. Within a medical or surgical setting, the outer diameters of the fitments within a series preferably vary from about 5 Fr. to about 40 Fr. Within a household or industrial plumbing setting, the outer diameters of the fitments within the series preferably vary from about ⅛ of an inch to about 6 inches or more.

As best illustrated in FIG. 2, the first and second outer ends 5A and 5B of the central housing 3 define at least one peripheral flange 85 about the housing's outer circumferential surface 6. Similarly, each tubular fitment defines at least one peripheral flange 85 about the fitment's outer circumferential surface (13; 23; 33; 43; 53; 63; and 73) proximal to the end (15; 25; 35; 45; 55; 65; and 75) of the fitment defining the internal threads. The at least one peripheral flange 85 of both the housing and fitments is configured for frictional engagement with the inner circumferential surface of the at least one segment of tubing. In the embodiment of FIGS. 2 and 3, the at least one peripheral flange 85 preferably has a cross-section defining a right triangle having its hypotenuse outwardly-directed away from the central housing 3. However, it is understood that the flange may define other cross sections as well, to include half-circles, squares, rectangles and other shapes understood as creating a frictional, fluid-tight fit with the interior circumferential surface of a tubing segment.

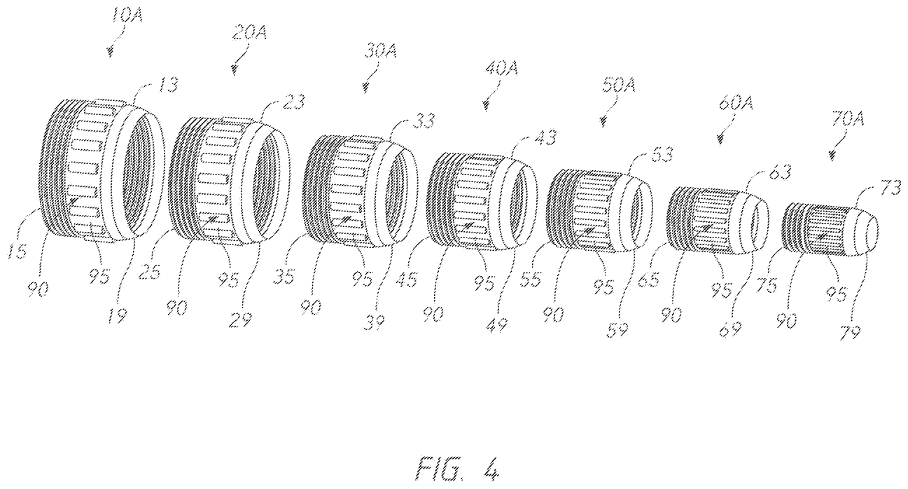

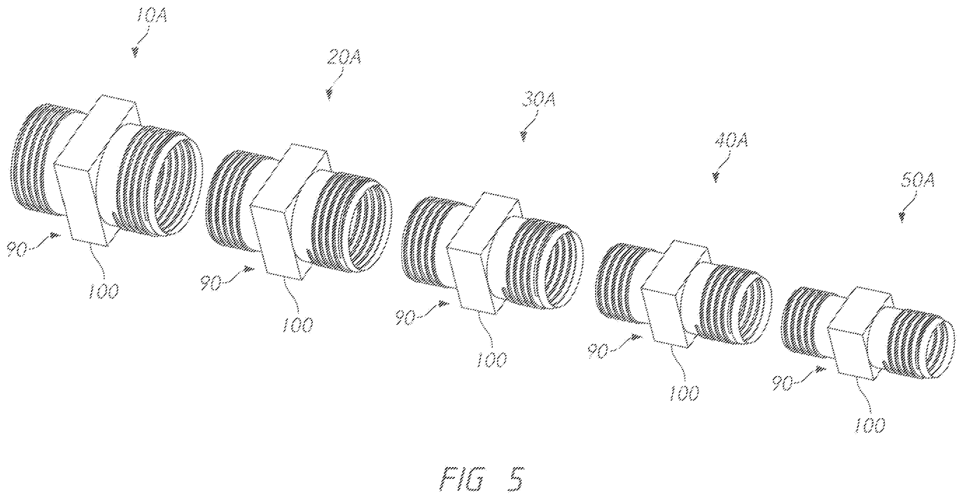

FIGS. 4 and 5 illustrate further embodiments of the fitments of the connector illustrated in FIGS. 2 and 3. As illustrated in FIGS. 4 and 5, the outer circumferential surface (13; 23; 33; 43; 53; 63; and 73) of at least one fitment defines a grip 90 between the fitment's respective first and second ends (15; 25; 35; 45; 55; 65; and 75) and (19; 29; 39; 49; 59; 69; and 79). For clarity, only the fitments of series A is illustrated in FIGS. 4 and 5. However, it is understood that the grips discussed herein are also defined on at least one fitment of series B as well. Similarly, in another embodiment, the central housing defines a grip on its outer surface between its outer ends. The grip aids in a grasping and rotating of the fitment and/or housing by a users' hand or tool in relation in relation to one another or the at least one tubing segment. To facilitate a grasping of the fitment by a hand of the user, the grip 90 preferably comprises a plurality of axial ridges 95 defined about the outer circumferential surface of each fitment (FIG. 4). It is understood however, that the grip may comprise a plurality of circumferal ridges, knurling, crosshatch or other textures as well. To facilitate a grasping of the fitment by a tool of the user, such as a common crescent wrench, the grip 90 preferably comprises a square 100 or hexagonal surface (not shown) defined about the outer circumferential surface of each fitment (FIG. 5).

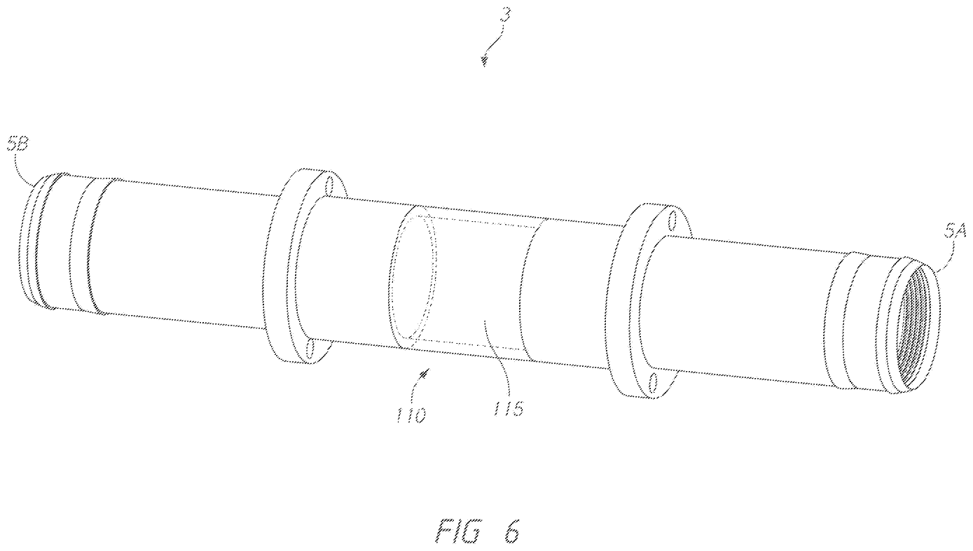

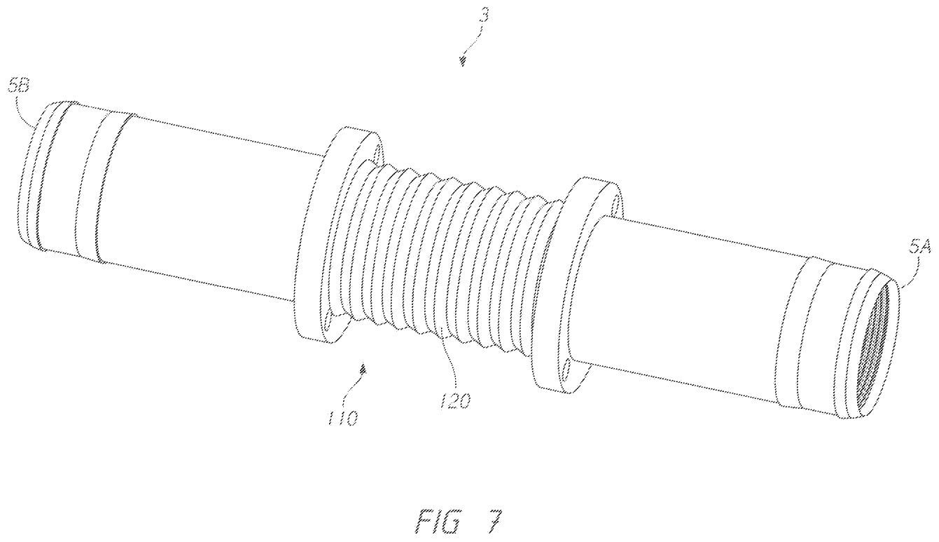

Referring now to FIGS. 6 and 7, an alternate embodiment of the central housing 3 of the connector 2 of FIGS. 2 and 3 defines an axially flexible portion 110 between the housing's first and second ends 5A and 5B. This flexible portion allows the central housing 3 to axially bend to alleviate any kinking that may occur within the at least one tubing segment connected to the connector's fitment(s). The flexible portion also allows the central housing to axially extend or contract between its outer ends to facilitate length adjustments (FIG. 7) of the connector assembly to accommodate it connection to tubing segments of extended of insufficient length. In one embodiment, the flexible portion 110 is comprised of an elastomeric tubular section 115 bonded between the two interior ends 15 and 19 of the housing 3 (FIG. 6). The elastomeric section 115 is preferably comprised of plastic, rubber, silicone etc. In another embodiment, the flexible portion 110 is comprised of an accordion section 120, preferably molded unitary with the housing 3, that exhibits a memory property to retain the flexible portion in a desired bent position. In further embodiments of the connector 2 (not shown), the foregoing flexible portions are defined in one of more of the fitments between the first and second ends.

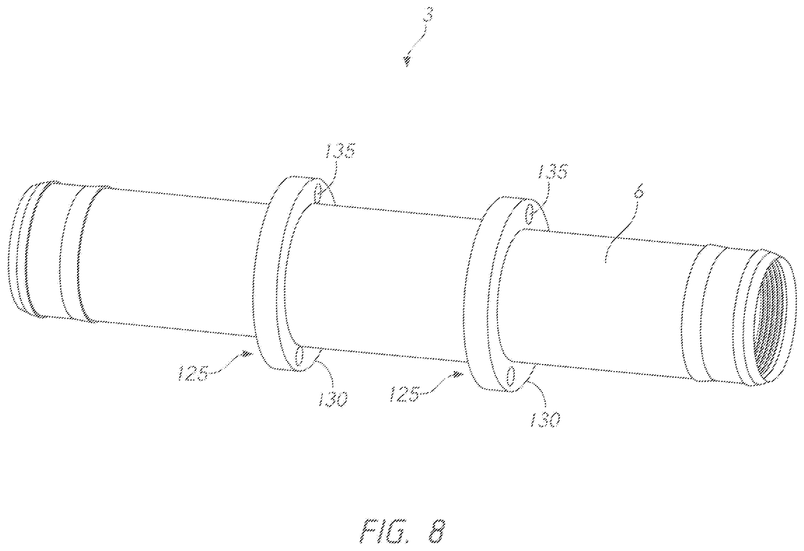

As illustrated in FIG. 8, other embodiments of the central housing 3 define at least one support 125 on its outer surface 6. The support 125, preferably comprising a peripheral circular ring 130 extending outwardly from the housing's outer surface 6, has at least one axial bore 135 defined there-through such that a wire, chain or string may be connected to the ring of the housing to facilitate a connection of the connector and attached at least one tubing segment to a hospital bed, IV stand or other component to prevent the connector and tubing segment from becoming contaminated from the floor or some other undesirable surface.

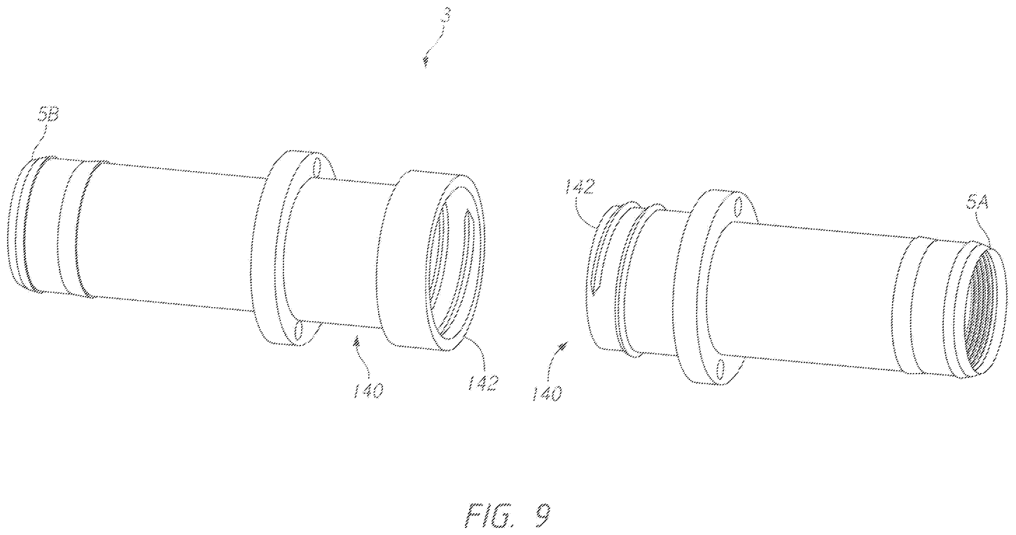

In yet another embodiment illustrated in FIG. 9, the central housing 3 defines a fluid-tight joint 140 between its outer ends 5A and 5B. This joint 140 allows for the housing to be disassembled such that the series of one or more fitments and/or tubular segments may be removably engaged to one or more of the outer ends 5A and 5B of the connector 2 during a medical or other application, and thereafter reassembled when the desired configuration of fitment(s)/tubular segment(s) is engaged therewith. The joint 140 preferably comprises a Luer lock 142 or other threaded, fluid-tight configuration. However, it is understood that other fluid-tight configurations understood in the art may be utilized as well.

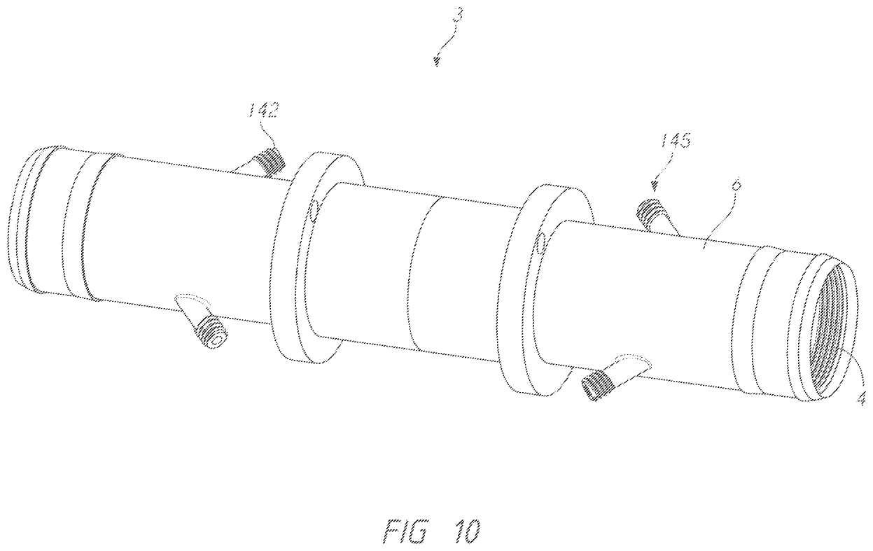

In a further embodiment of the central housing 3 illustrated in FIG. 10, the housing defines at least one access port 145 through the housing's outer surface 6 and in fluid communication with the at least one bore 4. The at least one access port 145 allows for the infusion of fluids or medications into the connector 2 and at least one tubing segment, or the extraction of blood or other fluids there-from for subsequent testing or laboratory analysis. To facilitate a connection of the at least one access port with a syringe, or with occluding cap that closes the port, an outer end of the port preferably defines a common Luer-lock fitting 142 or other threaded connection. Further details relating to the at least one access port are disclosed in applicant's co-pending U.S. patent application Ser. No. 17/714,961, which is fully incorporated herein. In yet a further embodiment (not shown), the central housing 3 defines a valve to occlude or regulate the axial flow of liquid there-through, to include a gate valve, globe valve, or other valve understood in the art

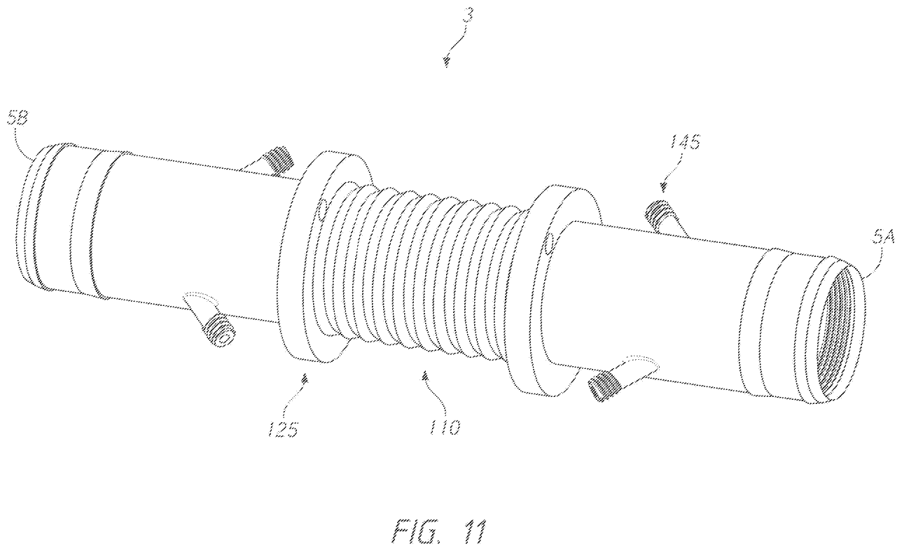

The housing 3 may define the foregoing components singularly or in combination with one or more of the other components. For example, as illustrated in FIG. 11, housing 3 simultaneously defines flexible portion 110, the at least one support 125, and the at least one access port 145 between its first and second ends 5A and 5B. It is further understood that various other combinations of these components is possible as well.

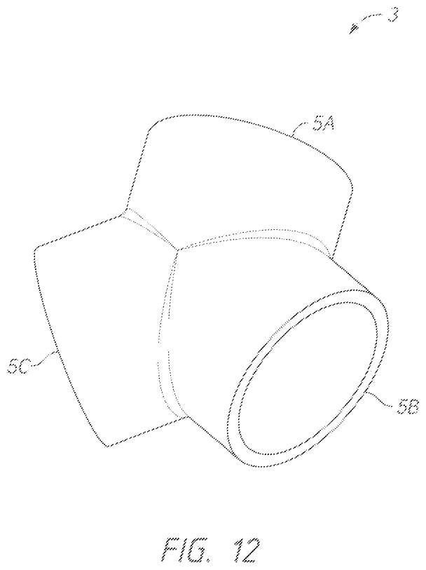

Referring now to FIG. 12, further embodiments of the connector 2 utilize a central housing 3 wherein the at least one bore 6 defines a “Y” configuration 150 to define first, second and third outer ends 5A, 5B and 5C of the central housing. As in previously discussed embodiments, these first, second and third ends 5A, 5B and 5C of the housing 3 are configured for axial removable engagement with respective first, second and third fitments of the plurality of tubular fitments, with each fitment of the preferably defining inner and outer surfaces having respective common inner and outer diameters and again axially aligned with the remaining fitments of the plurality about the housing in the descending order of the fitment's outer diameter. As also discussed in previous embodiments herein, each fitment of the series is removably engageable with an adjacent fitment and configured for fluid communication therewith.

In use, a central housing defining at least one through bore between at least first and second ends axially and removably engageable with at least a first pair of a plurality of paired tubular fitments is provided. Each fitment of a given pair defines a common inner and outer diameter and is axially aligned in opposition with one another in series relation about the housing in the descending order of the fitment's outer diameter to define a series of fitments. Each fitment of the series is also removably engaged with an adjacent fitment and in fluid communication therewith.

At least one tubing segment end defining a given inner diameter is also provided, after which a determination is made regarding which fitment of the series of fitments has an outer diameter sufficient to create a fluid-tight seal with the inner diameter of the tubing segment. Any fitment determined to have an outer diameter insufficient to create a fluid-tight seal with the inner diameter of the tubing segment is disengaged from the housing and connected series of fitments. An outer end of the fitment having the sufficient outer diameter is inserted into the at least one tubing segment end.

While this foregoing description and accompanying figures are illustrative of the present invention, other variations in system and method are possible without departing from the invention's spirit and scope.

We claim:

1. A size-specific tubing connector assembly for connecting at least one segment of tubing comprising: a central housing defining at least one through bore between at least first and second ends configured for axial removable engagement with at least a first pair of a plurality of tubular fitments, each fitment of the at least first pair defining a common inner and outer diameter and configured for axial alignment in opposition with one another in series relation about the housing in the descending order of the fitment's outer diameter to define a series of fitments, each fitment of the series removably engageable with an adjacent fitment.

2. The size-specific tubing connector assembly of claim 1 wherein the at least one bore of the central housing defines internal threads on an inner circumferential surface of the bore respectively proximal to the first and second ends, the first pair of the plurality of paired tubular fitments each defining external threads on the fitment's outer circumferential surface at a first end and internal threads on the fitment's inner circumferential surface at a second end, the external threads of each fitment of the first pair configured for threaded engagement with the internal threads of the housing, each tubular fitment of the remaining fitments of the series defining external threads on the fitment's outer circumferential surface at a first end and internal threads on the fitment's inner circumferential surface at a second end, the external threads of a given fitment configured for threaded engagement with the internal threads of an adjacent fitment within the series.

3. The size-specific tubing connector assembly of claim 2 wherein each tubular fitment defines at least one peripheral flange about the tubing's outer circumferential surface proximal to the end of the tubing defining the internal threads, the at least one peripheral flange configured for frictional engagement with the inner circumferential surface of the at least one segment of tubing.

4. The size-specific tubing connector assembly of claim 2 wherein the outer circumferential surface of each fitment defines a grip between the first and second ends, the grip selected from the group consisting of an axially ribbed outer surface, a square outer surface and a hexagonal outer surface as well as other shapes.

5. The size-specific tubing connector assembly of claim 1 wherein the central housing defines an axially flexible portion between the first and second ends.

6. The size-specific tubing connector assembly of claim 1 wherein the central housing defines a support between the first and second ends.

7. The size-specific tubing connector assembly of claim 1 wherein the central housing defines at least one access port in fluid communication with the bore.

8. The size-specific tubing connector assembly of claim 7 wherein an outer end of the access port defines a Luer-lock fitting.

9. The size-specific tubing connector assembly of claim 1 wherein the central housing and fitments are comprised of medical grade plastic or other possible material.

10. The size-specific tubing connector assembly of claim 2 wherein the outer diameter of each fitment differs in size from that of an adjacent fitment by an incremental value of at least 1 Fr.

11. The size-specific tubing connector assembly of claim 2 wherein the outer diameter of each fitment differs in size from an adjacent that of an adjacent fitment by an incremental value of at least 1 mm or 1/16 of an inch.

12. The size-specific tubing connector assembly of claim 10 wherein the outer diameters of the fitments are between about 5 Fr and about 40 Fr or more.

13. The size-specific tubing connector assembly of claim 10 wherein the outer diameters of the fitments are between about 1 mm and about 10 mm or more, or ⅛ of an inch to 6 inches or more.

14. The size-specific tubing connector assembly of claim 5 wherein the axially flexible portion comprises a rigid accordion segment to maintain a memory of a flexible position.

15. The size-specific tubing connector assembly of claim 1 wherein the at least one bore defines a “y-configuration” to define first, second and third outer ends of the central housing for respective axial removable engagement with a first, second and third fitments of a plurality of tubular fitments, each fitment of the three fitments defining a common inner and outer diameter and axially aligned in with the remaining fitments of the plurality about the housing in the order of the fitment's outer diameter, each fitment of the series removably engageable with an adjacent fitment and configured for fluid communication therewith.

16. A method of using a size-specific tubing connector assembly comprising:
providing a central housing defining at least one through bore between at least first and second ends axially and removably engaged with at least a first pair of a plurality of paired tubular fitments, each fitment of a given pair defining a common inner and outer diameter and axially aligned in opposition with one another in series relation about the housing in the descending order of the fitment's outer diameter to define a series of fitments, each fitment of the series removably engaged with an adjacent fitment and in fluid communication therewith;
providing at least one tubing segment end defining a given inner diameter;
determining which fitment of the series of fitments has an outer diameter sufficient to create a fluid-tight seal with the inner circumferential surface of the tubing segment;
disengaging any fitment from the series having an outer diameter insufficient to create a fluid-tight seal with the inner circumferential surface of the tubing segment; and
inserting an outer end of the fitment having the sufficient outer diameter into the at least one tubing segment end.

17. The method of claim 16 wherein the at least one bore of the central housing defines internal threads on an inner diameter of the bore respectively proximal to the first and second ends, the first pair of the plurality of paired tubular fitments each defining external threads on the fitment's outer diameter at a first end and internal threads on the fitment's inner diameter at a second end, the external threads of each fitment of the first pair configured for threaded engagement with the internal threads of the housing, each tubular fitment of the remaining fitments of the series defining external threads on the fitment's outer diameter at a first end and internal threads on the fitment's inner diameter at a second end, the external threads of a given fitment configured for threaded engagement with the internal threads of an adjacent fitment within the series.

18. The method of claim 17 wherein each tubular fitment defines at least one peripheral flange about the tubing's outer diameter proximal to the end of the tubing defining the internal threads, the at least one peripheral flange configured for frictional engagement with the inner diameter of the at least one segment of tubing.

19. The method of claim 17 wherein the outer diameter of each fitment defines a grip between the first and second ends, the grip selected from the group consisting of an axially ribbed outer surface and an outer surface defining a square, hexagonal or other shape.

20. The method of claim 16 wherein the central housing defines an axially flexible portion between the first and second ends.

21. The method of claim 16 wherein the central housing defines at least one access port in fluid communication with the bore.

22. The method of claim 17 wherein the outer diameter of each fitment differs in size from that of an adjacent fitment by an incremental value of at least 1 Fr or mm or ⅙ of an inch.
